(19) United States
(12) Patent Application Publication
(10) Pub. No.: US 2015/0060493 A1
(43) Pub. Date: Mar. 5, 2015

(54) FLUID PRODUCT DISPENSER

(71) Applicant: APTAR FRANCE SAS, Le Neubourg (FR)

(72) Inventors: Frédéric Duquet, Crespieres (FR); Simon Leoment, Paris (FR)

(73) Assignee: APTAR FRANCE SAS, Le Neubourg (FR)

(21) Appl. No.: 14/391,850

(22) PCT Filed: Jun. 6, 2013
(86) PCT No.: PCT/FR2013/051290

§ 371 Date: Oct. 10, 2014

(30) Foreign Application Priority Data
Jun. 7, 2012 (FR) 1255311

(51) Int. Cl. B05B 15/02; A61L 2/232; A61L 2/10; B05B 11/00

(52) U.S. Cl. CPC B05B 15/02; B05B 11/3001; A61L 2/232; A61L 2/10
USPC 222/148

(57) ABSTRACT

A fluid dispenser comprising a reservoir (1) and a dispenser member (2), the dispenser including at least one contact surface for coming into contact with the fluid, the fluid dispenser being characterized in that:
the contact surface includes a bactericidal photocatalyst; and
the dispenser includes a source (S) of radiation (R) for irradiating the bactericidal photocatalyst.

The present invention relates to a fluid dispenser comprising a dispenser member, such as a pump, having a fluid dispenser orifice where a user may collect the fluid that has been dispensed. The dispenser may also include a removable protective cap that masks the dispenser orifice in the storage condition, it being necessary to remove the cap in order to recover the fluid dispensed through the dispenser orifice. Advantageous fields of application of the present invention are the fields of cosmetics, perfumery, and pharmacy.

Dispensers of this type are very frequently used in the field of cosmetics for dispensing viscous fluids such as creams, gels, etc. The fluid leaves the dispenser orifice in the form of a bead or a glob. The user may recover the fluid by means of a finger, or onto another desired application surface. In the present invention, the dispenser orifice is any kind of end opening from which the fluid is accessible by the user. In particular, the dispenser orifice may be formed in a pusher that the user can move axially down by means of one or more fingers. The dispenser orifice often extends laterally or radially relative to the movement axis of the pusher. Thus, when the pusher is depressed, the dispenser orifice moves axially over a distance that corresponds to the stroke of the pusher. In order to prevent any unintentional or accidental actuation of the pusher and in order to protect the dispenser orifice, the pusher may be covered or masked by a protective cap that is generally in the form of an upsidedown cup. The user may press on the pusher so as to move it axially, thereby causing fluid to be dispensed through the dispenser orifice. Naturally, the user seeks to recover all of the fluid dispensed at the dispenser orifice. However, a residue of fluid always remains in the dispenser orifice and/or around the dispenser orifice. Given that it is extremely difficult to recover the residue of fluid, the user leaves the dispenser in this state. The dispenser orifice may thus be left dirty, and so also may other surfaces of the pusher and of the dispenser as a whole. As a result, the residue of fluid dries out and deteriorates, causing micro-organisms, germs, bacteria, microbes, etc. to appear. On the next dispensing operation, the user once again presses on the pusher so as to dispense the fluid through the dispenser orifice. The new dose of fluid naturally comes into contact with the dried-out and deteriorated residue of fluid resulting from the previous dispensing operation. As a result, the new dose of fluid also becomes contaminated by the micro-organisms, germs, bacteria, microbes, etc. resulting from the fluid residue. It is thus not guaranteed that the dispensed fluid is hygienic.

What is valid for a pusher optionally provided with a cap is also valid for other types of dispensers, such as pots, from which the user also collects the fluid at a dispenser orifice.

In a similar field, documents WO2007/018170 and WO2010/060979 make provision for sterilizing water vapor by means of TiO₂ irradiated with a lamp.

An object of the present invention is to remedy the above-mentioned drawback of the prior art by defining a dispenser having successive dispensed doses that are not contaminated by residues of fluid resulting from previous dispensing operations. Another object of the present invention is to avoid fundamentally overturning the structure of the dispenser. Still another object of the invention is to sterilize or to neutralize any residues of fluid without requiring any handling other than conventional handling, e.g. removing the cap from the dispenser orifice and putting it back into place.

To achieve these various objects, the present invention proposes a fluid dispenser comprising a reservoir and a dispenser member, such as a pump, the dispenser including at least one contact surface for coming into contact with the fluid, the contact surface including a bactericidal photocatalyst, the dispenser including a source of radiation for irradiating the bactericidal photocatalyst; the fluid dispenser being characterized in that the source of radiation is supported by a protective cap.

Thus, the function of the radiation coming from the protective cap is to activate or to stimulate the effect, or the bactericidal properties, of a photosensitive substance that in turn acts on the fluid present on the contact surfaces of the dispenser so as to sterilize or decontaminate them. The contact surface may be selected from a dispenser orifice, a pusher, a collection dish, a dispenser duct, etc.

In a practical embodiment, the dispenser member includes a fluid dispenser orifice via which the fluid is dispensed from the dispenser so as to be accessible to a user, the dispenser also including a removable protective cap that masks the dispenser orifice in the storage condition, the contact surface being defined at the dispenser orifice, the cap being provided with the source of radiation. The dispenser orifice may be formed in a collection dish, the contact surface extending in the dish. The dispenser orifice may be formed by a pusher, the contact surface extending over the pusher.

Advantageously, the source of radiation is arranged in the direct proximity of the contact surface.

In another aspect of the invention, the source of radiation incorporates trigger means for triggering the radiation, and timer means that are suitable for interrupting the radiation at the end of a determined period of time.

The radiation source may emit radiation, advantageously emitted by a light-emitting diode (LED), having a wavelength in the range about 280 nanometers (nm) to 380 nm, so as to activate the bactericidal effect of the photocatalyst. The bactericidal photocatalyst may be titanium dioxide TiO₂. The bactericidal photocatalyst is applied to the contact surface or is incorporated in a wall forming the contact surface.

One of the principles of the present invention is to use radiation that is appropriate for irradiating a bactericidal photocatalyst that sterilizes or de-contaminates any fluid residues present on the contact surfaces of the dispenser. However, this does not prevent the radiation from sterilizing or de-contaminating any fluid residues directly.

The invention is described more fully below with reference to the accompanying drawing, which shows an embodiment of the invention by way of non-limiting example.

In the figures:

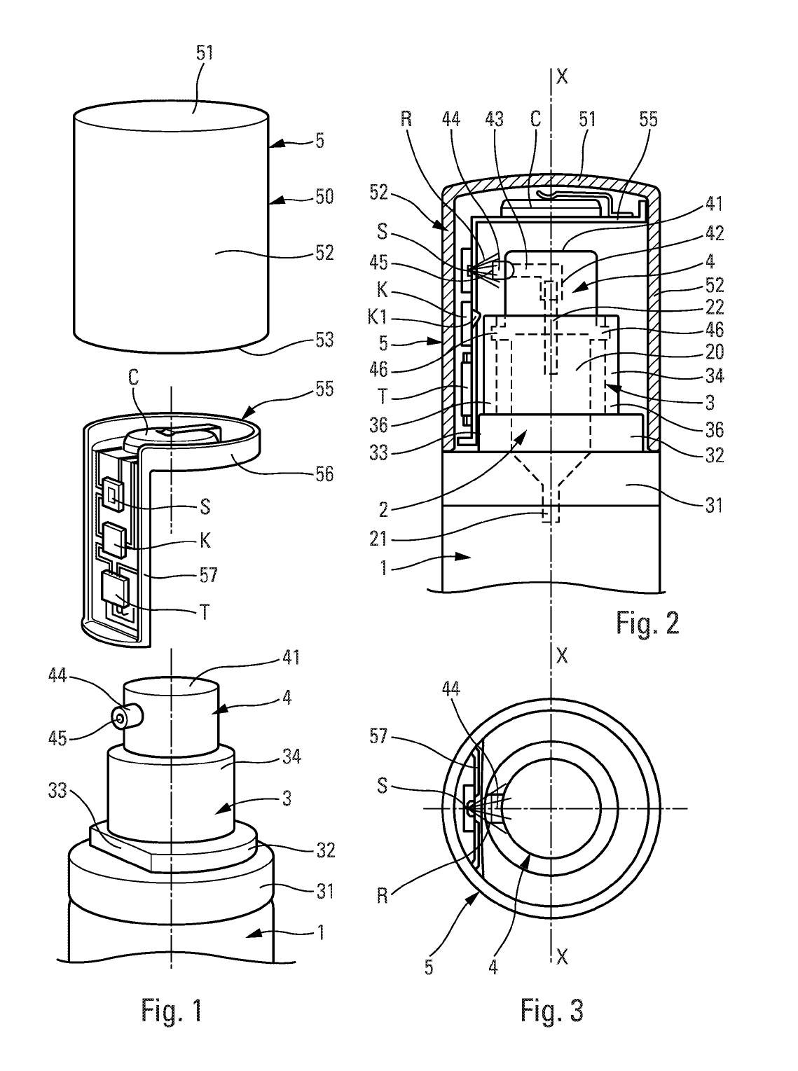

FIG. 1 is an exploded perspective view of a dispenser in a non-limiting embodiment of the invention;

FIG. 2 is a vertical section view through the FIG. 1 dispenser;

FIG. 3 is a horizontal cross-section view through the FIGS. 1 and 2 dispenser at the dispenser orifice; and

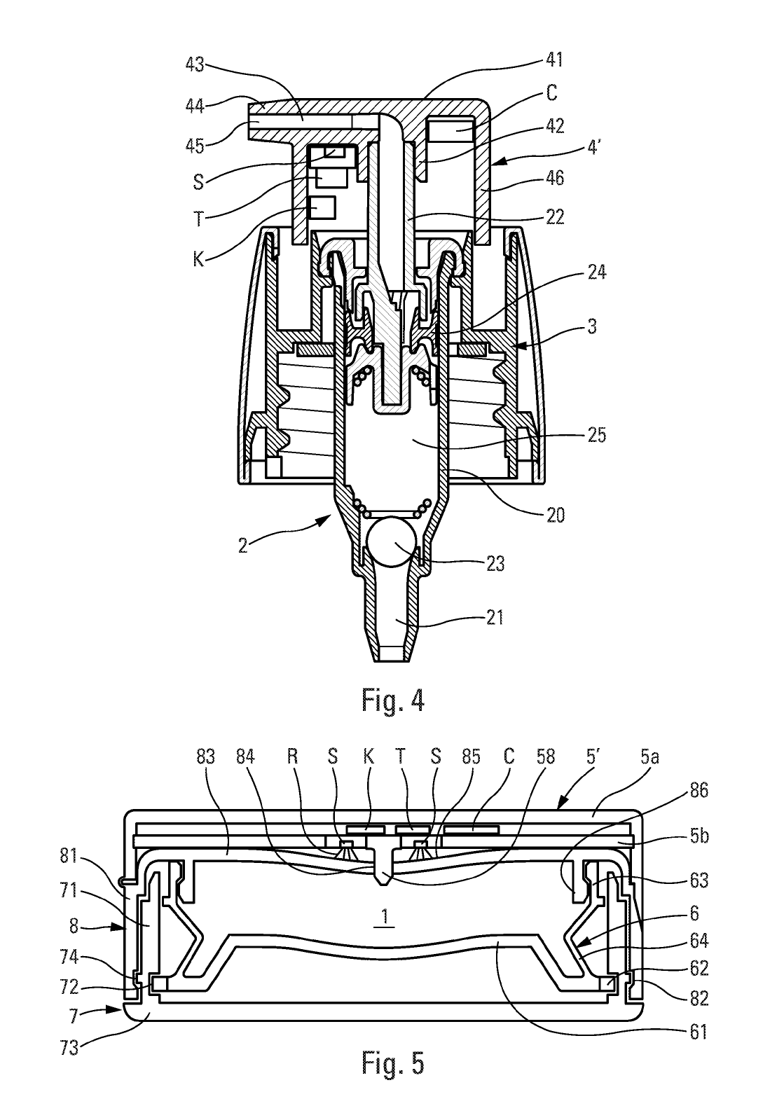

FIG. 4 is a vertical section view through a dispenser assembly constituting a second embodiment of the invention; and

FIG. 5 is a vertical section view through a dispenser constituting a third embodiment of the invention.

The fluid dispenser shown in the FIGS. 1 to 3 is more particularly for viscous fluids, such as creams, gels, etc. It essentially comprises five component elements, namely: a reservoir 1; a dispenser member 2, which is a pump; a fastener ring 3 for fastening the pump on the reservoir; a pusher 4 that is mounted on the pump; and a cap 5 that incorporates the present invention; except for the cap 5, and to a lesser extent the fastener ring 3, the other component elements, namely the reservoir 1, the pump 2, and the pusher 4 may be of design that is entirely conventional.

The fluid reservoir 1 may be of any kind, of any shape, and made of any appropriate material. It defines an internal fluid storage volume that may be constant or variable. In the field of cosmetics, variable-capacity reservoirs are generally used so that the fluid stored therein does not come into contact with the outside air. Naturally, the reservoir includes an opening that puts its internal volume into communication with the outside.

As mentioned above, the dispenser member 2 is a manual pump that includes a pump body 20 defining an inlet 21 that is in communication with the reservoir 1. The pump 2 also includes an actuator rod 22 that is axially movable down and up inside the body 20, in such a manner as to cause the volume of a pump chamber to vary so as to put a dose of fluid under pressure. In FIG. 2, it can be seen that the actuator rod 22 extends along the axial direction X, which also constitutes an axis of symmetry, or indeed an axis of revolution, for the dispenser. Although not shown, the actuator rod 22 is provided with a piston for sliding in leaktight manner inside a slide cylinder of the pump chamber. The operation of the pump is entirely conventional: by driving the actuator rod 22 into the pump body 20, the fluid contained in the pump chamber is put under pressure in such a manner as to be forced up through the actuator rod. When the actuator rod 22 is released, it returns to its rest position under the action of a return spring, and fluid from the reservoir is sucked into the pump chamber through the inlet 21.

The main function of the fastener ring 3 is to hold the pump 2 relative to the reservoir 1. Fastening is preferably permanent and leaktight. The fastener ring 3 comprises a bottom section 31 that engages the opening of the reservoir 1, an intermediate section 32, and a top section 34. In FIG. 1, it should be observed that the intermediate section 32 includes a flat 33 that interrupts its circular shape. As described below, the flat 33 serves to index the cap 5 relative to the remainder of the dispenser. The pump 2 is held, by any appropriate means, in stationary and leaktight manner inside the fastener ring 3. In FIG. 2, it should be observed that the pump body extends through the three sections of the fastener ring. The actuator rod 22 may project axially upwards out from the fastener ring 3.

The pusher 4 is mounted on the free end of the actuator rod 22, and may be moved downwards and upwards in the axial direction X. In this way, the pusher 4 drives the actuator rod 22 into the pump body 20. The pusher 4 includes a connection sleeve 42 that is engaged around the free end of the actuator rod 22, the sleeve communicating with an endpiece 44 via an internal delivery duct 43. The endpiece 44 forms a dispenser orifice 45 that can be seen in FIG. 1. It should be observed that the endpiece 44 projects radially or laterally outwards, such that the dispenser orifice 45 is oriented transversally, and more particularly perpendicularly, relative to the axial direction X. Thus, when the pusher 4 is moved axially downwards and upwards, the dispenser orifice 45 is also constrained to move with the pusher 4. While moving, the pusher 4 penetrates, in part, into the top section 34 of the fastener ring 3. In order to actuate the pusher, said pusher defines a bearing surface 41 on which the user can press by means of a finger. The pusher also includes a substantially-cylindrical side wall in which the endpiece 44 is formed.

At least a portion of the pusher 4 defines surfaces that may potentially come into contact with the fluid: in particular, said portion may be the dispenser orifice 45, the endpiece 44, the bearing surface, and/or the side wall. In the invention, the contact surfaces include a bactericidal photocatalyst: said bactericidal photocatalyst may be applied to the surfaces or incorporated in a wall that forms the contact surfaces. The bactericidal photocatalyst may be TiO₂. TiO₂ reacts to radiation having a wavelength in the range about 280 nm to 380 nm which amplifies or activates the bactericidal effect of the photocatalyst.

The protective cap 5 includes a cap body 50 that is preferably opaque. The cap body 50 presents a general configuration in the shape of an upsidedown cup, thus defining a top wall 51 and a substantially-cylindrical side wall 52 that defines an annular bottom edge 53. Once in place on the dispenser, as shown in FIG. 2, the top wall 51 is arranged above the pusher 4, and the side wall 52 extends around the pusher 4 and the fastener ring 3. More precisely, the side wall 52 surrounds the intermediate section 32 and the top section 34 of the fastener ring: the bottom annular edge 53 of the cap 5 coming to bear on the bottom section 31 of the fastener ring 3. Snap-fastening may be provided between the cap 5 and the fastener ring 3, so as to hold the cap 5 securely on the dispenser in its storage condition.

The above-described dispenser presents a design that is entirely conventional in the fields of cosmetics, perfumery, and pharmacy. The pusher 4, and more particularly its dispenser orifice 45, associated with the cap 5 constitute a fluid dispenser head in the broadest sense. Without going beyond the ambit of the invention, it is possible to disassociate the dispenser orifice from the pusher 4, e.g. so as to make the orifice stationary relative to the reservoir 1. The dispenser orifice 45 may thus be connected to the pusher 4 via a flexible hose. Other dispenser configurations also make it possible to disassociate the dispenser orifice from the pusher 4. In the context of the present invention, the dispenser head should be understood as the association of a dispenser orifice with a protective cap. In the particular non-limiting embodiment shown in the figures, the dispenser head is constituted by the pusher 4 and the cap 5.

In the invention, the protective cap 5 includes a support element 55 that is arranged inside the space formed by the cap. By way of example, the support element 55 may form a top plate 56 and a side tab 57 that are connected together at an edge of the plate, such that the tab 57 extends axially downwards. Thus, the support element 55 may be inserted inside the cap body 50 so that the plate 56 comes to be positioned immediately below the top wall 51, and the tab 57 immediately against the side wall 52. This is clearly visible in FIG. 2 and also understandable from FIG. 1. The bottom end of the tab 57 extends down substantially as far as the bottom end of the side wall 52 of the cap 5, so as to make it possible to position it against the flat 33 of the intermediate section 32 of the fastener ring 3. Thus, the support element 55 constrains the protective cap 5 to take up a particular angular orientation relative to the fastener ring 3, and as a result relative to the remainder of the dispenser. The cap 5 is thus always oriented in the same way relative to the dispenser. In addition, the pusher 4 is prevented from turning relative to the remainder of the dispenser, such that the endpiece 44 and its dispenser orifice 45 are always oriented in the same way. By way of example, in order to block the pusher 4, it is possible to provide two guide lugs 46 on the pusher 4 that slide in two axial grooves 36 that are formed in the top section 34 of the fastener ring 3. This is a conventional characteristic that is frequently used to prevent the pusher from turning. Thus, the cap 5 is indexed relative to the dispenser and the dispenser orifice 45 is prevented from turning, thereby constraining the dispenser orifice 45 to occupy a particular position relative to the cap 5. As can be seen in FIGS. 2 and 3, the dispenser endpiece 44 is oriented towards the tab 57 that has a bottom end that comes into contact with the flat 33. This constitutes the only possible position for the cap 5 relative to the dispenser and the dispenser orifice.

In the invention, the support element 55 supports a radiation source S that is suitable for emitting radiation R for irradiating the contact surfaces of the pusher that may potentially come into contact with the fluid being dispensed. In this way, when any residual fluid remains, e.g. at the dispenser orifice and/or around the dispenser orifice, it is decontaminated and/or sterilized by the bactericidal photocatalyst, which presents a bactericidal effect that needs to be catalyzed or activated by the radiation R. It can be seen in FIGS. 2 and 3 that the source S and its radiation R are arranged in the proximity of, and immediately facing, the dispenser orifice 45. The source S is arranged on a printed circuit card that is mounted on the tab 57. In order to activate the source S, trigger means K are provided, e.g. in the form of a switch including a trigger member K1 that may be mechanical and/or electronic. By way of example, provision may be made for the trigger member K1 to come into contact with the top section 34 of the fastener ring 3. The trigger member K1 may also be provided in the form of a presence detector that detects the presence of an object inside the cap. The trigger member K1 may thus function with or without direct contact. The trigger means K are advantageously associated with timer means T that are suitable for interrupting the radiation R at the end of a determined period of time. By way of example, the timer means may act on the trigger means K so as to reinitialize them. By way of example, it is possible to provide radiation duration of about 10 seconds to about 1 minute. For electrically powering the system, it is possible to provide a battery C that may be arranged at the plate 56. Once the support element 55 is equipped in this way, it is inserted into the cap body 50, with all of the electronic elements arranged between the support element 55 and the cap body so that no electronic element can be seen.

The protective cap 5 is handled in exactly the same way as a conventional protective cap. It is put into place and removed by moving it axially in the direction X. When it is put into place, the trigger member K1 detects the presence of the pusher 4 and/or of the fastener ring 3, or, in a variant, it comes into direct contact with the pusher 4 and/or the fastener ring 3, so as to trigger radiation R from the source S. During this operation, the user does not intervene in any way in order to trigger and operate the source S. The radiation R is thus emitted for a determined period of time by the timer means T. At the end of this period of time, the radiation R is stopped. The protective cap 5 then once again provides no more than a conventional protection function. When the user removes the cap 5 axially, the source S remains inactive: it is only while the cap is being put back into place on the dispenser that the source emits its radiation once again for a determined period of time. Consequently, the cap is handled in entirely conventional manner: The user may even be unaware of the presence of the radiation source and of the associated electronic components.

In an application of the present invention, the radiation R is radiation that has a wavelength in the range about 280 nm to 380 nm, that is suitable for performing a catalyzing function for the bactericidal photocatalyst which may be TiO₂. By way of example, the radiation may be emitted by an LED. The radiation may also participate directly in destroying the bacteria present in the fluid. Naturally, the intensity depends on the power of the source S, on the distance from the source to the orifice, and on the irradiation time of the radiation R. The fluid residues that accumulate at the dispenser orifice and/or around the dispenser endpiece 44 are thus decontaminated and/or sterilized, such that the next-dispensed dose of fluid is not contaminated by the fluid residues resulting from prior dispensing operations.

In the above-described embodiment, the radiation source S is carried by the cap 5. In a variant that is not shown, it is also possible to envisage positioning the source remotely in another component element of the dispenser, such as the fastener ring 3 for example, and to convey the radiation by means of a waveguide or an optical fiber to the dispenser orifice. The same applies for the associated electronic elements, such as the trigger means K and the timer means T, and the battery C, that could be housed inside the fastener ring 3. A principle of the invention is to use the cap 5 as a support for supporting radiation emitted directly onto the dispenser orifice and its close surroundings. Another principle is to use the radiation to activate or to amplify the bactericidal effect of a bactericidal photocatalyst.

FIG. 4 shows a dispenser assembly that is ready to be mounted on a reservoir, thereby constituting a fluid dispenser. The assembly comprises a pump 2 having a pump body 20, an inlet 21, an actuator rod 22, an inlet valve 23, and a piston 24 that co-operate with each other to define a conventional pump chamber. The pump is provided with a fastener ring 3 and a pusher 4′. As in the first embodiment, the pusher is mounted on the free end of the actuator rod 22, and may be moved downwards and upwards in the axial direction X. In this way, the pusher 4 drives the actuator rod 22 into the pump body 20. The pusher 4 includes a connection sleeve 42 that is engaged around the free end of the actuator rod 22, the sleeve communicating with an endpiece 44 via an internal delivery duct 43. The endpiece 44 forms a dispenser orifice 45 that can be seen in FIG. 1. It should be observed that the endpiece 44 projects radially or laterally outwards, such that the dispenser orifice 45 is oriented transversally, and more particularly perpendicularly, relative to the axial direction X. In order to actuate the pusher, said pusher defines a bearing surface 41 on which the user can press by means of a finger. The pusher also includes a substantially-cylindrical side wall 46 in which the endpiece 44 is formed.

In the invention, the pusher 4′ contains at least one source S of radiation R, a battery C, trigger means K for triggering radiation, and timer means T that are suitable for interrupting the radiation R at the end of a determined period of time, as in the above-described embodiment. All of these elements may be housed inside the pusher. Advantageously, the source S emits its radiation R directly towards the duct 43 and the bearing surface 41. Advantageously, the material constituting the pusher is, at least locally, transparent to the radiation R, so that it can diffuse through the mass of the pusher. In addition, the material constituting the pusher contains a bactericidal photocatalyst, such as TiO₂, that is applied to its surface, or is preferably embedded in its mass. The radiation, having a wavelength in the range about 280 nm to 380 nm, activates or amplifies the bactericidal effect of the photocatalyst that acts on any fluid residues present on the pusher, in particular at the dispenser orifice 45, the endpiece 44, and also at the bearing surface 41 or the side wall 46.

In this second embodiment, the pusher is used to diffuse the radiation that catalyzes the bactericidal photocatalyst. Such a diffuser pusher may also be used in the first embodiment.

Reference is made below to FIG. 5, which shows a fluid dispenser in the form of a pot that essentially comprises four component elements, namely a bellows 6, actuator means 7, a shell 8, and a protective cap or cover 5′.

By way of example, the bellows 6 is a single piece made out of a deformable flexible material such as an elastomer or a thermoplastic elastomer. It presents good deformability properties in very specific zones. The bellows 1 includes a bottom wall 61 that is substantially rigid. On its outer periphery, the bottom wall 61 defines a plurality of drive lugs 62 that are substantially rigid. The bellows 6 also forms a fastener collar 63 that is also substantially rigid. The bellows 1 also includes a deformable wall 64 that connects the fastener collar 63 to the bottom wall 61. Thus, it is possible to move the wall 61 in translation closer to the fastener collar 63 by deforming the wall 64. More precisely, the deformable wall 64 forms a fold that is adapted to become more pronounced when pressure is exerted that tends to move the collar 63 closer to the lugs 62. It is precisely the deformable wall 64 that gives the part its name, since it imparts a bellows function thereto. By way of example, the bellows 6 may be replaced by a flexible pouch that is fastened on a pouch support performing the function of fastener collar 63. The bottom wall 61 may be replaced by a plate against which the flexible pouch comes to bear.

The actuator means 7 include a bushing 71 that is internally threaded in such a manner as to form one or more internal threads 72. As can be seen in FIG. 5, the drive lugs 62 of the bellows are in threaded engagement with the internal threads 72. Below the threaded bushing 71, the actuator means define a bottom wall 73 that serves as an actuator and grip element that the user may grip so as to turn the actuator means. On the outer wall of the threaded bushing 20, a continuous bead or a plurality of projecting bead segments 74 is/are formed extending over a fraction of the entire periphery of the bushing 71. It can easily be understood that while the bellows 6 is held stationary, turning the actuator means 7 causes the lugs 62 to move inside the threaded bushing 71.

The body 8 of the dispenser forms a kind of outer shell that, to a large extent, gives the dispenser its attractive appearance. The body 8 includes a peripheral skirt 81 inside which the bushing 71 of the actuator means 7 is arranged. Internally, the skirt 81 is formed with an annular housing 82 in which the projecting bead segments 74 that are formed on the outside of the threaded bushing 71 are received. Engaging the bead segments 74 in the housing 82 holds the bushing 71 free to turn inside the skirt 81. Thus, the actuator means 7 may turn freely in the skirt 81, but cannot become disengaged therefrom. Above the skirt 81, the body 8 forms a plate 83 in which a hole is formed that serves as a dispenser orifice 84 for the dispenser. The orifice 84 is advantageously situated in a collection dish 85 formed by the plate 83. A connection sleeve 86 extends below the plate 83 and is engaged with the leaktight fastener collar 63 of the bellows 6. Thus, the bellows 6 is connected in stationary manner, with the body 8 being prevented even from turning. As a result, the bellows 6 and the body 8 co-operate with each other to form a reservoir of volume that varies given that the movable wall 61 may move towards the plate 83 as a result of the wall 64 deforming. By way of example, the user may grip the body 8 in the left hand, and turn the actuator means 7 in the right hand by gripping its bottom wall 73. This causes the drive lugs 62 to rise in the internal threads of the bushing 72. The movable wall 61 thus rises towards the plate 83, reducing the working volume of the reservoir 1. The fluid stored inside the reservoir is thus driven through the dispenser orifice 84 so as to enable it to be collected by the user in the collection dish 85.

The cover or cap 5′ may be connected integrally to the body 8 via a bridge of material. The cover 5′ includes a top plate 5a and a bottom plate 5b that is fitted in the dish so as to form a housing between them containing at least one source S of radiation R, a battery C, trigger means K for triggering the radiation, and timer means T that are suitable for interrupting the radiation R at the end of a determined period of time, as in the above-described embodiments. The bottom plate 5b includes as many openings as there are sources of radiation R, so as to direct the radiation towards the dish 85 and the dispenser orifice 84. On its bottom face, the bottom plate 5b defines a closure pin 58 that is adapted to become housed in leaktight manner in the dispenser orifice 84 so as to close it hermetically. This occurs when the cover 5′ is folded down on the plate 83. Radiation from the source S may thus also irradiate a portion of the pin 58, and even a portion of the bottom plate 5b.

In addition, the dish 85, the orifice 84, the pin 58, and/or the plate 5b which define all of the surfaces that may potentially come into contact with the fluid, include a bactericidal photocatalyst that is applied to their surfaces or incorporated in their mass. Either way, the radiation R from the source(s) irradiates the contact surface(s) of the element(s) where the bactericidal photocatalyst is present, thereby causing the bactericidal effect to be activated. The bactericidal photocatalyst may be TiO₂, as in the above-described embodiments.

In all of the embodiments, the radiation from the source is used to activate, amplify, or stimulate the bactericidal properties of a sensitive substance such as TiO₂. The combination of appropriate radiation and a bactericidal photocatalyst may be applied to any surface of a fluid dispenser. However, the radiation could also have a direct bactericidal action on the fluid. However, it should be observed that for a source of radiation having a direct bactericidal effect, its wavelength should lie in the range 250 nm to 300 nm (UVC spectrum), ideally being 254 nm. The cost of the light sources remains high and their efficiency is low in the margin 250 nm to 280 nm.

The invention consists in using a source with a longer wavelength (>280 nm) placed in the UVB and UVA spectrum, and activating the bactericidal effect of an additive contained in the plastics material, e.g. of the pusher. Specifically, the additive used is titanium dioxide (TiO₂). The bactericidal effect of TiO₂ is increased when it is subjected to irradiation of wavelength that is shorter than 385 nm.

Producing sources of light at wavelengths lying in the range 280 nm to 380 nm is less costly, and their efficiency is greater than the efficiencies obtained at wavelengths that have a direct bactericidal effect (254 nm).

1.-9. (canceled)

10. A fluid dispenser comprising a reservoir and a dispenser member including a fluid dispenser orifice via which the fluid is dispensed from the dispenser so as to be accessible to a user, the dispenser including at least one contact surface for coming into contact with the fluid, the contact surface including a bactericidal photocatalyst, the dispenser further including a source of radiation for irradiating the bactericidal photocatalyst, the dispenser including a removable protective cap that masks the dispenser orifice in the storage condition;
the fluid dispenser being characterized in that the source of radiation is supported by a protective cap, the contact surface being defined at the dispenser orifice.

11. A dispenser according to claim 10, wherein the contact surface is selected from:
a dispenser orifice;
a pusher;
a collection dish; and
a dispenser duct.

12. A dispenser according to claim 10, wherein the dispenser orifice is formed in a collection dish, the contact surface extending in the dish.

13. A dispenser according to claim 10, wherein the dispenser orifice is formed by a pusher, the contact surface extending over the pusher.

14. A dispenser according to claim 10, wherein the source of radiation is arranged in the direct proximity of the contact surface.

15. A dispenser head according to claim 10, wherein the source of radiation incorporates trigger means for triggering the radiation, and timer means that are suitable for interrupting the radiation at the end of a determined period of time.

16. A dispenser head according to claim 10, wherein the radiation source emits radiation, advantageously emitted by an LED, having a wavelength in the range about 280 nm to 380 nm, so as to activate the bactericidal effect of the photocatalyst.

17. A dispenser head according to claim 10, wherein the bactericidal photocatalyst is titanium dioxide TiO₂.

18. A dispenser head according to claim 10, wherein the bactericidal photocatalyst is applied to the contact surface or is incorporated in a wall forming the contact surface.
